(19) United States
(12) Patent Application Publication
(10) Pub. No.: US 2008/0027575 A1
(43) Pub. Date: Jan. 31, 2008

(54) Modeling systems for health and beauty consumer goods

(76) Inventors: Stevan David Jones, Cincinnati, OH (US); William David Laidig, Hamilton, OH (US); David Thomas Stanton, Hamilton, OH (US); Bryan Patrick Murphy, Loveland, OH (US); James Charles Dunbar, Morrow, OH (US); Bruce Prentiss Murch, Cincinnati, OH (US); John David Shaffer, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/787,818

(22) Filed: Apr. 18, 2007

Related U.S. Application Data
(60) Provisional application No. 60/793,899, filed on Apr. 21, 2006.

(51) Int. Cl. G06F 19/00

(52) U.S. Cl. 700/97

(57) ABSTRACT

The present invention relates to modeling systems for designing consumer beauty care products and selected components for use in consumer beauty care products, consumer beauty care products and components selected by such models and the use of same.

### CROSS REFERENCE TO RELATED APPLICATION

This application claims the benefit of U.S. Provisional Application No. 60/793,899, filed Apr. 21, 2006.

### FIELD OF THE INVENTION

The present invention relates to modeling systems for designing consumer products, and in particular health and beauty consumer products, and selected components for use in such consumer products and components selected by such models and the use of same. Application of embodiments of the modeling system enables one to gain a superior understanding of the role of the components in the form of the final health and beauty product performance attributes, in delivery of component-beauty and/or component-health substrate interactions, in delivery of component-health and/or component-beauty packaging interactions, and in delivery of the final targeted health and/or beauty performance.

### BACKGROUND OF THE INVENTION

Health and beauty consumer goods are typically designed and/or formulated using empirical methods or basic modeling methodologies. Such efforts are time consuming, expensive and, in the case of empirical methodologies, generally do not result in optimum designs/formulations as not all components and parameters can be considered. In addition, nor is there typically a solid understanding of the role of health and/or beauty product components, particularly regarding the following: a) interactions with the specific beauty substrate area, b) packaging interactions, c) the delivery of the overall target health and/or beauty performance, and d) basic component stability within the product's stability. Furthermore, aspects of such methods may be limited to existing components. Thus there is a need for an effective and efficient methodology that obviates the short comings of such methods.

### SUMMARY OF THE INVENTION

The present invention relates to modeling systems for designing beauty and/or health consumer products and selected beauty and/or health components for use in consumer products, and components selected by such models and the use of same. Embodiments of the modeling techniques described herein enable delivery of superior understanding of the role of such components in component-beauty and/or component-health substrate interactions, component-beauty and/or component-health packing interactions, and in the final delivery of the targeted product performance.

Embodiments of modeling systems of the present invention meet the aforementioned desires and, in addition, can be used to define component parameters, such as dependent and independent properties, that can be used to produce new and superior formulation components related to beauty and/or health product(s), including without limitation: Health and Beauty Performance Attributes (e.g., including without limitation hair shine, hair condition, skin feel, skin whiteness, hair volume/body, tooth whiteness, lip softness, lip fullness, eye lash thickness, breath freshness, skin condition, skin tone, skin naturalness, etc.); Sensory Areas (e.g., including without limitation touch, taste, smell, visual, sound); Consumer Sensory Attributes (e.g., including without limitation shampoo creaminess, cream whiteness, toothpaste flavor (e.g., minty-ness), lather creaminess/whiteness/rinseability, styling gel stickiness/hold, mascara durability, foundation greasiness, lipstick durability, etc.); Product Stability (e.g., including without limitation light stability, temperature stability, stability to metal ions, etc.); Package Performance Attributes (e.g., including without limitation ease of actuation, ease of grip, package breatheability, spray characteristics (low versus high particle size, wet versus dry, etc.), etc.), and Package Stability (e.g., including without limitation erosion stability, pressure stability, etc.).

Embodiments of the modeling techniques described herein can be used to generate superior technical understanding of typical health and/or beauty components (and the dependent and independent properties of such components) in the delivery of targeted health and/or beauty product performances (e.g., shampoo creaminess, cream whiteness, toothpaste minty-ness, lather creaminess/whiteness/rinseability, styling gel stickiness/hold, mascara durability, foundation greasiness, lipstick durability, hair cleanliness, shine, condition, color, skin color, whiteness, greasiness, dryness, lip gloss durability, shine, anti-dandruff, antiperspirant efficacy, etc.).

Embodiments of the modeling techniques described herein can be used to generate superior technical understanding of typical beauty-involved substrates (hair, skin, teeth, lashes, lips, etc.) and/or typical health-involved substrates.

Embodiments of the modeling techniques described herein can be used to generate superior technical understanding of typical beauty-component and/or health-component (and the dependent and independent properties of such component) and interactions of such component with typical beauty-involved and/or health-involved substrates (hair, skin, teeth, lashes, lips, etc.).

Embodiments of the modeling techniques described herein can be used to generate superior technical understanding of typical beauty-component and/or health-component (and the dependent and independent properties of such components) and interactions of such component with typical beauty-involved and/or health-involved packaging (bottles, jars, cans, aerosol and non-aerosol sprays, etc.).

Embodiments of the modeling techniques described herein can be used to generate superior technical understanding of typical beauty-component and/or health-component (and the dependent and independent properties of such components) and interactions of such components in product related stability (e.g., including without limitation light stability, temperature stability, stability to metal ions, etc.).

### DETAILED DESCRIPTION OF THE INVENTION

### Definitions

As used herein “consumer products” includes, without limitation unless otherwise indicated, baby care, beauty care, fabric & home care, fiber, paper, family care, feminine care, health care, pet care, snack and/or beverage products or devices commercially available for use by consumers. Specifically, “beauty consumer products” includes, without limitation unless otherwise indicated, lipsticks, lip paints, lip softeners, eye mascaras, eye shadows, eye pencils, eye lash thickeners, face cosmetics (rouges, foundations, creams, coverers), skin creams, lotions & milks, skin cleansers, skin whiteners, skin feel, skin moisturizers, exfoliants, anti-acne agents, sun protectors, hair sprays, hair volumizers and bodifiers, hair gels, hair mousses, hair waxes, hair tonic, shampoos, conditioners, soap bars, body washes, shine agents, dyes, bleaches, perming agents, straightening agents, sun protectants, anti-dandruff shampoos, antiperspirants, deodorants, toothpaste, and tooth whitening agents. In some instances, the products are intended to be used or consumed in the form in which they are sold, and are not intended for subsequent commercial manufacture or modification. However, in some instances, the consumer products may be modified, assembled, combined with other consumer products and/or components and/or otherwise manufactured to some degree by a consumer. Such components include but are not limited to specific molecules or molecular structures and/or compounds and/or mixtures of molecules and/or compounds.

Such products include but are not limited to home décor, batteries, diapers, bibs, wipes; products for and/or methods relating to treating hair (human, dog, and/or cat), including bleaching, coloring, dyeing, conditioning, shampooing, styling; deodorants and antiperspirants; paper products; pet care products; health care products; beauty care products; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee and cleaning and/or treatment compositions.

More specifically, in some instances, the health and/or beauty products are intended to be used or consumed in the form in which they are sold and are not intended for subsequent commercial manufacture or modification. However, in some instances, the health and/or beauty products may be modified, assembled, combined with other consumer products, and/or otherwise manufactured to some degree by a consumer.

As used herein, the term “acute toxicity” includes, where applicable, but is not limited to acute terrestrial toxicity, acute reproductive and developmental toxicity, acute neurotoxicity, acute respiratory toxicity, acute phototoxicity, acute endocrine toxicity, hepatotoxicity, acute cardiovascular toxicity, acute renal toxicity, acute immunotoxicity, acute hematotoxicity, acute gastrointestinal toxicity, acute oral toxicity, acute nasal toxicity, and acute musculoskeletal toxicity for all living species, including but not limited to microbes and mammals, for example humans.

As used herein, the term “chronic toxicity” includes, where applicable, but is not limited to chronic terrestrial toxicity, chronic reproductive and developmental toxicity, chronic neurotoxicity, chronic respiratory toxicity, chronic phototoxicity, chronic endocrine toxicity, hepatotoxicity, chronic cardiovascular toxicity, chronic renal toxicity, chronic immunotoxicity, chronic hematotoxicity, chronic gastrointestinal toxicity, chronic oral toxicity, chronic nasal toxicity, and chronic musculoskeletal toxicity for all living species, including but not limited to microbes and mammals, for example humans.

As used herein the term “non-polymer consumer product component” does not include polymers.

As used herein, the term “situs” includes paper products, fabrics, garments and hard surfaces.

As used herein, the articles “a” and “an” when used in a claim, are understood to mean one or more of what is claimed or described.

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

Modeling Methods

In a first aspect, Applicant's modelling method comprises:
a.) correlating a dependent property of an initial beauty and/or health consumer product component, with an independent variable of said component; said step employing typically comprising:
   (i) structure entry into a computer, said structure entry can be achieved via sketching using, for example, the following software such as: Sybyl® (Ver. 6.9, Tripos, Inc, St. Louis, Mo.); Cerius2® (Ver. 4.9, Accelrys, Inc., San Diego, Calif.); ChemFinder™ (Ver. 7.0, CambridgeSoft, Cambridge, Mass.); Spartan '02 (Build 119, Wavefunction, Inc., Irvine, Calif.); CAChe™ (Ver. 5.0, Fujitsu America, Sunnyvale, Calif.); JME Molecular Editor©, or reading pre-stored structures, suitable non-limiting storage formats include SMILES strings; MDL® CTfile or SDF file, Tripos MOL and MOL2 file, PDB file, HyperChem® HIN file, CAChe™ CSF file,;
   (ii) generating 3D atomic coordinates as needed, said generation optionally employing a technique selected from the group consisting of 2D-3D converters, conformational analysis, conformational optimization or combination thereof, and can be achieved using, for example Concord® (Tripos, Inc, St. Louis, Mo.); Corina (Molecular Networks GmbH, Erlangen, Germany); Omega (OpenEye Scientific Software, Santa Fe, N. Mex.); Cerius2® (Ver. 4.9, Accelrys, Inc., San Diego, Calif.); Chem3D™ (Ver. 7.0, CambridgeSoft, Cambridge, Mass.); Spartan '02 (Build 119, Wavefunction, Inc., Irvine, Calif.); CAChe™ (Ver. 5.0, Fujitsu America, Sunnyvale, Calif.), AMPAC™(Ver. 7.0, Semichem, Shawnee Mission, Kans.), Hyperchem® (Ver. 7.5, Hypercube, Inc., Gainsville, Fla.);
   (iii) calculating said independent variable, said calculation being achieved in one aspect of said method by using, for example, Cerius2® (Ver. 4.9, Accelrys, Inc., San Diego, Calif.); Spartan '02 (Build 119, Wavefunction, Inc., Irvine, Calif.); CAChe™ (Ver. 5.0, Fujitsu America, Sunnyvale, Calif.), Codessa™ (Ver. 2.7.2, Semichem, Shawnee Mission, Kans.); ADAPT (Prof. P. C. Jurs, Penn State University, University Park, Pa.); Dragon (Talete, srl., Milano, Italy); Sybyl® (Ver. 6.9, Tripos, Inc, St. Louis, Mo.), MolconnZ™ (Ver. 4.05, eduSoft, Ashland, Va.), Hyperchem® (Ver. 7.5, Hypercube, Inc., Gainsville, Fla.);
   (iv) performing objective feature analysis as needed, said objective feature analysis typically including removing independent variables exhibiting little or no variance and/or removing independent variables showing high pairwise correlation to other independent variables; said performance can be achieved by employing, for example, ADAPT (Prof. P. C. Jurs, Penn State University, University Park, Pa.); Minitab® (Ver. 14, Minitab, Inc., State College, Pa.); JMP™ (Ver. 5.1, SAS Institute Inc., Cary, N.C.); Mobydigs (Talete, srl., Milano, Italy);
   (v) generating a statistical model (e.g., molecular model) that correlates said dependent property with said independent variable such generation achieved in one aspect of said method by employing, for example, Cerius2® (Ver. 4.9, Accelrys, Inc., San Diego, Calif.); CAChe™ (Ver. 5.0, Fujitsu America, Sunnyvale, Calif.), Codessa™ (Ver. 2.7.2, Semichem, Shawnee Mission, Kans.); ADAPT (Prof. P. C. Jurs, Penn State University, University Park, Pa.); Sybyl® (Ver. 6.9, Tripos, Inc, St. Louis, Mo.); Minitab® (Ver. 14, Minitab, Inc., State College, Pa.); JMP™ (Ver. 5.1, SAS Institute Inc., Cary, N.C.); Mobydigs (Talete, srl., Milano, Italy); Simca-P (Umetrics, Inc. Kinnelon, N.J.); R Statistical Language (The R Foundation for Statistical Computing); S-Plus® (Insightful®, Seattle, Wash.);
b.) calculating said dependent property for an additional beauty and/or health consumer product component by inputting said independent variable of said additional consumer product component into the correlation of Step a.); and/or defining the relationship between changes in said initial component's structure (e.g., molecular structure) and said initial component's dependent property by analysing the correlation of Step a.);
c.) optionally, using the output of Step b.) to refine the correlation of Step a.); and
d.) optionally repeating Steps a.) through c.).

In certain embodiments, the structure entered into the computer is a molecular structure of a component within the consumer product. However, the structure of a consumer product may be analyzed at a different level of granularity, and thus may not be a molecular structure in other embodiments.

In said first aspect of the modelling method, said correlation may be achieved by employing a technique selected from the group consisting of multiple linear regression, genetic function method, generalized simulated annealing, principal components regression, non-linear regression, projection to latent structures regression, neural networks, support vector machines, logistic regression, ridge regression, cluster analysis, discriminant analysis, decision trees, nearest-neighbor classifier, molecular similarity analysis, molecular diversity analysis, comparative molecular field analysis, Free and Wilson analysis, and combinations thereof; a technique selected from the group consisting of multiple linear regression, genetic function method, generalized simulated annealing, principal components regression, non-linear regression, projection to latent structures regression, neural networks, support vector machines, logistic regression, ridge regression, cluster analysis, discriminant analysis, molecular similarity analysis, molecular diversity analysis, and combinations thereof; or even more simply a technique selected from the group consisting of multiple linear regression, genetic function method, generalized simulated annealing, projection to latent structures regression, neural networks, cluster analysis, discriminant analysis, molecular similarity analysis, molecular diversity analysis, and combinations thereof.

In said first aspect of the modelling method, said initial beauty and/or health consumer product component may be selected from the group consisting of surfactants, chelating agents, cleansing agents, emulsion stabilizers, oils, fixative and/or thickening/viscosity modifying and/or conditioning and/or film-forming agents and polymers, anti-oxidants and radical scavengers, dyes and pigments, opacifying agents, exfoliants, UV absorbers and reflectors, conditioning agents, shine enhancers, slip agents, perfumes and fragrances, flavoring agents, solvents and solubilizers, hydrotopes, preservatives and anti-bacterials and anti-fungal agents, humectants, astringents, depilatory agents, pH adjusting and buffering agents, anti-static agents, bleaching agents, anti-dandruff agents, antiperspirant and deodorants, anti-acne, anti-foaming agents, foam boosters, hair waving and/or straightening agents, hair and teeth and skin bleaching/whitening and coloring agents, oxidizing and reducing agents, corrosion inhibitors, aerosol and non-aerosol propellants, plasticizers, suspending agents, enzymes and enzyme stabilizers, dye transfer inhibiting agents, dispersants, and enzyme stabilizers, catalysts, bleach activators, sources of hydrogen peroxide, preformed peracids, brighteners, dyes, perfumes, carriers, hydrotropes, solvents and combinations thereof.

In one aspect of the modelling method said initial beauty consumer product component is not a polymer having a solubility of at least 10 ppm at 20° C., a weight average molecular weight from about 1500 to 200,000 daltons comprising a main chain and at least one side chain extending from the main chain; the side chain comprising an alkoxy moiety and the side chain comprising a terminal end such that the terminal end terminates the side chain. In one or more aspects of the modelling method said initial consumer product component is a non-polymer component. In one or more aspects of the modelling method said initial consumer product component is a biological material such as a protein and/or sugar based component, such as cellulose. In at least one aspect, the initial beauty consumer product component is at least one component of a beauty care or other consumer product.

In certain embodiments, the dependent property is selected from the group consisting of: concentration; partition coefficient; vapor pressure; solubility; permeability; permeation rate; chemical reaction, including but not limited to atmospheric degradation and/or transformation, hydrolysis, and photolysis; color; color intensity; color bandwidth; CIE Lab color definition; solubility parameters; particle size; light transmission; light absorption; coefficient of friction; color change; viscosity; phase stability; pH; ultraviolet spectrum; visible light spectrum; infrared spectrum; vibrational frequency; Raman spectrum; circular dichroism; nuclear magnetic resonance spectrum; mass spectrum; boiling point; melting point; freezing point; chromatographic retention index; refractive index; surface tension; surface coverage; critical micelle concentration; odor detection threshold; odor character; human odor-emotive response; protein binding; bacterial minimum inhibition concentration; enzyme inhibition concentration; enzyme reaction rate; host-guest complex stability constant; receptor binding; receptor activity; ion-channel activity; ion concentration; molecular structure similarity; mutagenicity; carcinogenicity; acute toxicity; chronic toxicity; skin sensitization; irritations, including but not limited to eye, oral, nasal and skin irritations; absorption; distribution; metabolism; excretion; Type I allergy; bioconcentration; biodegradation, including but not limited to, biodegradation metabolite maps; bioaccumulation; Henrys Law constants; Phase Type and Properties; and combinations thereof.

In said first aspect of the modelling method, said dependent property may be selected from the group consisting of component: concentration; partition coefficient; vapor pressure; solubility; permeability; permeation rate; chemical reaction, including but not limited to atmospheric degradation and/or transformation, hydrolysis, and photolysis; color; color intensity; color bandwidth; CIE Lab color definition; solubility parameters; particle size; light transmission; light absorption; coefficient of friction; color change; viscosity; phase stability; pH; ultraviolet spectrum; visible light spectrum; infrared spectrum; vibrational frequency; Raman spectrum; circular dichroism; nuclear magnetic resonance spectrum; mass spectrum; boiling point; melting point; freezing point; chromatographic retention index; refractive index; surface tension; surface coverage; critical micelle concentration; odor detection threshold; odor character; human odor-emotive response; protein binding; bacterial minimum inhibition concentration; enzyme inhibition concentration; enzyme reaction rate; host-guest complex stability constant; receptor binding; receptor activity; ion-channel activity; ion concentration; molecular structure similarity; mutagenicity; carcinogenicity; acute toxicity; chronic toxicity; skin sensitization; irritations, including but not limited to eye, oral, nasal and skin irritations; absorption; distribution; metabolism; excretion; Type I allergy; bioconcentration, biodegradation, bioaccumulation, including biodegradation metabolite maps; Henrys Law constants; and combinations thereof; said dependent property may be selected from the group consisting of component: concentration, partition coefficient, vapor pressure, solubility, permeability, permeation rate, chemical reaction, color, color intensity, color bandwidth, CIE Lab color definition, solubility parameters, particle size, light transmission, light absorption, coefficient of friction, color change, viscosity, phase stability, pH, boiling point, melting point, freezing point, chromatographic retention index, refractive index, surface tension, critical micelle concentration, odor detection threshold, odor character, human odor-emotive response, bacterial minimum inhibition concentration, enzyme inhibition concentration, enzyme reaction rate, host-guest complex stability constant, molecular structure similarity, mutagenicity, carcinogenicity, acute toxicity, chronic toxicity, skin sensitization, and combinations thereof; or even more simply said dependent property may be selected from the group consisting of component: concentration, partition coefficient, vapor pressure, solubility, permeability, permeation rate, chemical reaction, color, color intensity, color bandwidth, CIE Lab color definition, solubility parameters, light transmission, light absorption, coefficient of friction, color change, viscosity, phase stability, pH, boiling point, melting point, freezing point, chromatographic retention index, refractive index, surface tension, critical micelle concentration, odor detection threshold, odor character, bacterial minimum inhibition concentration, host-guest complex stability constant, molecular structure similarity; Phase Type (not limited to but including gas, liquid, solid, wax, gel) and Properties; and combinations thereof.

According to certain embodiments, the dependent property may be one or more components included in a consumer product. According to certain embodiments, the dependent property may comprise any one or more of the following types of properties: Consumer Performance Attributes (e.g., hair shine, hair condition, skin feel, skin whiteness, hair volume/body, tooth whiteness, lip softness, lip fullness, eye lash thickness, breath freshness, skin condition, skin tone, skin naturalness, etc.); Consumer Sensory Areas (e.g., touch, taste, smell, visual, sound); Consumer Product Sensory Attributes (e.g., shampoo creaminess, cream whiteness, toothpaste flavor (e.g., minty-ness), lather creaminess/whiteness/rinseability, styling gel stickiness/hold, mascara durability, foundation greasiness, lipstick durability, etc.); Product Stability (e.g., light stability, temperature stability, stability to metal ions, etc.); Package Performance Attributes (e.g., ease of actuation, ease of grip, package breatheability, spray characteristics (low versus high particle size, wet versus dry, etc.), etc.); and Package Stability (e.g., erosion stability, pressure stability, etc.).

In said first aspect of the modelling method, said independent variable may be selected from the group consisting of constitutional descriptors, Hammett parameters, substituent constants, molecular holograms, substructure descriptors, BC(DEF) parameters, molar refractivity, molecular polarizability, topological atom pairs descriptors, topological torsion descriptors, atomic information content, molecular connectivity indices, electrotopological-state indices, path counts, Kier molecular shape descriptors, distance connectivity indices, Wiener index, centric indices, flexibility descriptors, molecular identification numbers, information connectivity indices, bond information index, molecular complexity indices, resonance indices, van der Waals surface area and volume, solvent-accessible surface area and volume, major moments of inertia, molecular length, width, and thickness, shadow areas, through-space distance between atoms and molecular fragments, radius of gyration, 3D-Weiner index, volume overlaps, sterimol parameters, geometric atom pairs descriptors, chirality descriptors, cis/trans descriptors, dipole and higher moments, resonance indices, hydrogen-bonding descriptors, partial atomic charges, HOMO energy level, LUMO energy level, electrostatic potential, quantum-chemical hardness and softness indices, superdelocalizability indices, ionization potential, molecular fields, excited state energies, polarizability, hyperpolarizability, charged partial surface area descriptors, hydrophobic surface area descriptors, Burden eigenvalues, BCUT descriptors, molecular docking scores, binding constants, octanol-water partition coefficient, cyclohexane-water partition coefficient, normal boiling point, chromatographic retention indices, nuclear magnetic resonance spectra, infrared spectra, ultraviolet spectra, color (visible wavelength) spectra, pKa, aqueous solubility, Hansen solubility parameters, Hoy solubility parameters, heat of formation, heat of vaporization, protein-ligand binding, protein receptor activation, protein receptor inhibition, enzyme inhibition, skin permeability, hydrophobic-hydrophilic balance, and combinations thereof; said independent variable may be selected from the group consisting of constitutional descriptors, substituent constants, molecular holograms, substructure descriptors, molar refractivity, molecular polarizability, molecular connectivity indices, electrotopological-state indices, path counts, Kier molecular shape descriptors, distance connectivity indices, Wiener index, centric indices, flexibility descriptors, molecular identification numbers, bond information index, molecular complexity indices, van der Waals surface area and volume, solvent-accessible surface area and volume, major moments of inertia, molecular length, width, and thickness, radius of gyration, volume overlaps, chirality descriptors, cis/trans descriptors, dipole moments, resonance indices, hydrogen-bonding descriptors, partial atomic charges, HOMO energy level, LUMO energy level, electrostatic potential, quantum-chemical hardness and softness indices, superdelocalizability indices, ionization potential, charged partial surface area descriptors, hydrophobic surface area descriptors, binding constants, octanol-water partition coefficient, pKa, aqueous solubility, Hansen solubility parameters, hydrophobic-hydrophilic balance, and combinations thereof; or even more simply said independent variable may be selected from the group consisting of constitutional descriptors, substituent constants, substructure descriptors, molar refractivity, molecular polarizability, molecular connectivity indices, electrotopological-state indices, path counts, Kier molecular shape descriptors, distance connectivity indices, Wiener index, flexibility descriptors, molecular identification numbers, molecular complexity indices, van der Waals surface area and volume, solvent-accessible surface area and volume, major moments of inertia, molecular length, width, and thickness, radius of gyration, dipole moments, hydrogen-bonding descriptors, partial atomic charges, HOMO energy level, LUMO energy level, electrostatic potential, quantum-chemical hardness and softness indices, superdelocalizability indices, charged partial surface area descriptors, hydrophobic surface area descriptors, octanol-water partition coefficient, pKa, aqueous solubility, and combinations thereof. In one or more aspects of the aforementioned model, COSMO-R descriptors are not employed as an independent variable.

According to certain embodiments, the independent variable may be one or more components included in a consumer product. According to certain embodiments, the independent variable may comprise any one or more of the following types of variables: Consumer Performance Attributes (e.g., hair shine, hair condition, skin feel, skin whiteness, hair volume/body, tooth whiteness, lip softness, lip fullness, eye lash thickness, breath freshness, skin condition, skin tone, skin naturalness, etc.); Consumer Sensory Areas (e.g., touch, taste, smell, visual, sound); Consumer Product Sensory Attributes (e.g., shampoo creaminess, cream whiteness, toothpaste flavor (e.g., minty-ness), lather creaminess/whiteness/rinseability, styling gel stickiness/hold, mascara durability, foundation greasiness, lipstick durability, etc.); Product Stability (e.g., light stability, temperature stability, stability to metal ions, etc.); Package Performance Attributes (e.g., ease of actuation, ease of grip, package breatheability, spray characteristics (low versus high particle size, wet versus dry, etc.), etc.); and Package Stability (e.g., erosion stability, pressure stability, etc.).

In an aspect of the aforementioned modelling method, said dependent property may be selected from the group consisting of component: concentration, partition coefficient, vapor pressure, solubility, permeability, permeation rate, reaction rate, color, color intensity, solubility parameters, particle size, light transmission, light absorption, coefficient of friction, color change, viscosity, phase stability, pH, ultraviolet spectrum, visible light spectrum, infrared spectrum, nuclear magnetic resonance spectrum, mass spectrum, boiling point, melting point, freezing point, chromatographic retention index, refractive index, surface tension, surface coverage, critical micelle concentration, odor detection threshold, odor character, human odor-emotive response, protein binding, bacterial minimum inhibition concentration, enzyme inhibition concentration, enzyme reaction rate, host-guest complex stability constant, receptor binding, receptor activity, ion-channel activity, ion concentration, molecular structure similarity, mutagenicity, carcinogenicity, acute toxicity, chronic toxicity, skin sensitization, rate of metabolism, rate of excretion, and combinations thereof; and said independent variable may be selected from the group consisting of constitutional descriptors, Hammett parameters, substituent constants, molecular holograms, substructure descriptors, BC(DEF) parameters, molar refractivity, molecular polarizability, topological atom pairs descriptors, topological torsion descriptors, atomic information content, molecular connectivity indices, electrotopological-state indices, path counts, Kier molecular shape descriptors, distance connectivity indices, Wiener index, centric indices, flexibility descriptors, molecular identification numbers, information connectivity indices, bond information index, molecular complexity indices, resonance indices, van der Waals surface area and volume, solvent-accessible surface area and volume, major moments of inertia, molecular length, width, and thickness, shadow areas, through-space distance between atoms and molecular fragments, radius of gyration, 3D-Weiner index, volume overlaps, sterimol parameters, geometric atom pairs descriptors, chirality descriptors, cis/trans descriptors, dipole moments, resonance indices, hydrogen-bonding descriptors, partial atomic charges, HOMO energy level, LUMO energy level, electrostatic potential, quantum-chemical hardness and softness indices, superdelocalizability indices, ionization potential, molecular fields, charged partial surface area descriptors, hydrophobic surface area descriptors, Burden eigenvalues, BCUT descriptors, molecular docking scores, binding constants, octanol-water partition coefficient, cyclohexane-water partition coefficient, normal boiling point, chromatographic retention indices, nuclear magnetic resonance spectra, infrared spectra, ultraviolet spectra, color (visible wavelength) spectra, pKa, aqueous solubility, Hansen solubility parameters, heat of formation, heat of vaporization, protein binding, skin permeability, hydrophobic-hydrophilic balance, and combinations thereof.

In an aspect of said modelling method, said dependent property may be selected from the group consisting of component: concentration, partition coefficient, vapor pressure, solubility, permeability, permeation rate, reaction rate, color, color intensity, solubility parameters, light transmission, light absorption, coefficient of friction, color change, viscosity, phase stability, pH, boiling point, melting point, freezing point, chromatographic retention index, refractive index, surface tension, critical micelle concentration, odor detection threshold, odor character, bacterial minimum inhibition concentration, host-guest complex stability constant, molecular structure similarity, and combinations thereof; said independent variable may be selected from the group consisting of constitutional descriptors, substituent constants, substructure descriptors, molar refractivity, molecular polarizability, molecular connectivity indices, electrotopological-state indices, path counts, Kier molecular shape descriptors, distance connectivity indices, Wiener index, flexibility descriptors, molecular identification numbers, molecular complexity indices, van der Waals surface area and volume, solvent-accessible surface area and volume, major moments of inertia, molecular length, width, and thickness, radius of gyration, dipole moments, hydrogen-bonding descriptors, partial atomic charges, HOMO energy level, LUMO energy level, electrostatic potential, quantum-chemical hardness and softness indices, superdelocalizability indices, charged partial surface area descriptors, hydrophobic surface area descriptors, octanol-water partition coefficient, pKa, aqueous solubility, and combinations thereof; and said correlation may be achieved by employing a technique selected from the group consisting of multiple linear regression, projection to latent structures regression, neural networks, cluster analysis, discriminant analysis, molecular similarity analysis, molecular diversity analysis, and combinations thereof.

In any of the foregoing aspects of the invention said dependent property may be single dependent property, the output of Step b.) may be used to refine the correlation of Step a.); Steps a.) through c.) may be repeated at least once; the output of Step b.) may be used to refine the correlation of Step a.) or combination thereof.

In another aspect of the invention, when the consumer product component is a polymer, modelling may be conducted as previously described except the correlation Step a.) is achieved using a technique other than multiple linear regression, or the correlation technique does not employ molecular fragmentation.

Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with perfumes, colorants, dyes, or the like. It is understood that such adjuncts are in addition to the dye conjugate and optional stripping agent components of Applicants' compositions. The precise nature of these additional components, and levels of incorporation thereof, will depend on the physical form of the composition and the nature of the cleaning operation for which it is to be used. Suitable adjunct materials include, but are not limited to, surfactants, builders, chelating agents, cleansing agents, emulsion stabilizers, oils, fixative and/or thickening/viscosity modifying and/or conditioning and/or film-forming agents and polymers, anti-oxidants and radical scavengers, dyes and pigments, opacifying agents, exfoliants, UV absorbers and reflectors, conditioning agents, shine enhancers, slip agents, perfumes and fragrances, flavoring agents, solvents and solubilizers, hydrotopes, preservatives and anti-bacterials and anti-fungal agents, humectants, astringents, depilatory agents, pH adjusting and buffering agents, anti-static agents, bleaching agents, anti-dandruff agents, anti-perspirant and deodorants, anti-acne, anti-foaming agents, foam boosters, hair waving and/or straightening agents, hair and teeth and skin bleaching/whitening and coloring agents, oxidizing and reducing agents, corrosion inhibitors, aerosol and non-aerosol propellants, plasticizers, suspending agents, enzymes and enzyme stabilizers, and combinations thereof. In addition to the disclosure below, suitable examples of such other adjuncts and levels of use are found in U.S. Pat. Nos. 5,576,282, 6,306,812 B1, and 6,326,348 B1 that are incorporated by reference.

As stated, the adjunct ingredients are not essential to Applicants' compositions. Thus, certain embodiments of Applicants' compositions do not contain one or more of the following adjuncts materials: surfactants, builders, chelating agents, cleansing agents, emulsion stabilizers, oils, fixative and/or thickening/viscosity modifying and/or conditioning and/or film-forming agents and polymers, anti-oxidants and radical scavengers, dyes and pigments, opacifying agents, exfoliants, UV absorbers and reflectors, conditioning agents, shine enhancers, slip agents, perfumes and fragrances, flavoring agents, solvents and solubilizers, hydrotopes, preservatives and anti-bacterials and anti-fungal agents, humectants, astringents, depilatory agents, pH adjusting and buffering agents, anti-static agents, bleaching agents, anti-dandruff agents, anti-perspirant and deodorants, anti-acne, anti-foaming agents, foam boosters, hair waving and/or straightening agents, hair and teeth and skin bleaching/whitening and coloring agents, oxidizing and reducing agents, corrosion inhibitors, aerosol and non-aerosol propellants, plasticizers, suspending agents, enzymes and enzyme stabilizers, and combinations thereof. However, when one or more adjuncts are present, such one or more adjuncts may be present as detailed below:

Bleaching Agents—Bleaching agents other than bleaching catalysts include photobleaches, bleach activators, hydrogen peroxide, sources of hydrogen peroxide, preformed peracids. Examples of suitable bleaching agents include anhydrous sodium perborate (mono or tetra hydrate), anhydrous sodium percarbonate, tetraacetyl ethylene diamine, nonanoyloxybenzene sulfonate, sulfonated zinc phtalocyanine and mixtures thereof.

When a bleaching agent is used, the compositions of the present invention may comprise from about 0.1% to about 50% or even from about 0.1% to about 25% bleaching agent by weight of the subject cleaning composition.

Surfactants—The compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof.

The surfactant is typically present at a level of from about 0.1% to about 60%, from about 1% to about 50% or even from about 5% to about 40% by weight of the subject composition.

Chelating Agents—The compositions herein may contain a chelating agent. Suitable chelating agents include copper, iron and/or manganese chelating agents and mixtures thereof.

When a chelating agent is used, the composition may comprise from about 0.1% to about 15% or even from about 3.0% to about 10% chelating agent by weight of the subject composition.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof.

When present in a subject composition, the dye transfer inhibiting agents may be present at levels from about 0.0001% to about 10%, from about 0.01% to about 5% or even from about 0.1% to about 3% by weight of the composition.

Enzymes—The compositions can comprise one or more enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, mannanases, pectate lyases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is an enzyme cocktail that comprises a protease, lipase, cutinase and/or cellulase in conjunction with amylase.

When present in a cleaning composition, the aforementioned adjunct enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. In case of aqueous compositions comprising protease, a reversible protease inhibitor can be added to further improve stability.

Solvents—Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

Processes of Making Cleaning and/or Treatment Compositions

The cleaning compositions of the present invention can be formulated into any suitable form and prepared by any process chosen by the formulator, non-limiting examples of which are described in Applicants examples and in U.S. Pat. Nos. 5,879,584; 5,691,297; 5,574,005; 5,569,645; 5,565,422; 5,516,448; 5,489,392; and 5,486,303 all of which are incorporated herein by reference.

Method of Use

The consumer products of the present invention may be used in any conventional manner. In short, they may be used in the same manner as consumer products that are designed and produced by conventional methods and processes. For example, cleaning and/or treatment compositions of the present invention can be used to clean and/or treat a situs inter alia a surface or fabric. Typically at least a portion of the situs is contacted with an embodiment of Applicants' composition, in neat form or diluted in a wash liquor, and then the situs is optionally washed and/or rinsed. For purposes of the present invention, washing includes but is not limited to, scrubbing, and mechanical agitation. The fabric may comprise any fabric capable of being laundered in normal consumer use conditions. Cleaning solutions that comprise the disclosed cleaning compositions typically have a pH of from about 5 to about 10.5. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 100:1.

The exemplary modelling methods described herein may be employed for modelling any number of consumer products, including without limitation those consumer products described in any of the following U.S. Patents, the disclosures of which are hereby incorporated herein by reference:

The exemplary modelling methods described herein may be employed for virtual product/technology development and/or virtual combinatorial chemistry for beauty products. The exemplary modelling methods may employ a virtual library design method, virtual screening method (e.g., to screen for particular components and/or attributes of a consumer product being modelled), and/or virtual high throughput screening method.

Exemplary Composition Forms

The topical compositions of exemplary beauty consumer products that may be modelled using the modelling techniques described herein can include, but are not limited to: Lipsticks, lip paints, lip softeners, eye mascaras, eye shadows, eye pencils, eye lash thickeners, face cosmetics (rouges, foundations, cremes, coverers), skin creams, lotions & milks, skin cleansers, skin whiteners, skin feel, skin moisturizers, exfoliants, anti-acne agents, sun protectors, hair sprays, hair volumizers & bodifiers, hair gels, hair mousses, hair waxes, hair tonic, shampoos, conditioners, soap bars, body washes, shine agents, dyes, bleaches, perming agents, straightening agents, sun protectants, anti-dandruff shampoos, antiperspirants, deodorants, toothpaste, tooth whitening agents. Such cosmetic products may include conventional ingredients such as cleansing agents, emulsion stabilizers, oils, fixative and/or thickening/viscosity modifying and/or conditioning and/or film-forming agents and polymers, anti-oxidants and radical scavengers, dyes and pigments, opacifying agents, exfoliants, UV absorbers and reflectors, conditioning agents, shine enhancers, slip agents, perfumes and fragrances, flavoring agents, solvents and solubilizers, hydrotopes, preservatives and anti-bacterials and anti-fungal agents, humectants, astringents, depilatory agents, pH adjusting and buffering agents, anti-static agents, bleaching agents, anti-dandruff agents, antiperspirant and deodorants, anti-acne, anti-foaming agents, foam boosters, hair waving and/or straightening agents, hair and teeth and skin bleaching/whitening and coloring agents, oxidizing and reducing agents, corrosion inhibitors, aerosol and non-aerosol propellants, plasticizers, suspending agents, enzymes and enzyme stabilizers, dye transfer inhibiting agents, dispersants, and enzyme stabilizers, catalysts, bleach activators, sources of hydrogen peroxide, preformed peracids, brighteners, dyes, perfumes, carriers, hydrotropes, solvents and combinations thereof. Exemplary carriers and such other ingredients which can be suitable for use herein are described, for example, in U.S. Pat. No. 6,060,547.

Methods for Regulating Keratinous Tissue Condition

The exemplary modelling techniques described herein may be employed to determine/evaluate compositions that are useful for regulating a number of mammalian keratinous tissue conditions. Such regulation of keratinous tissue conditions includes prophylactic and therapeutic regulation. More specifically, such regulating methods are directed to, but are not limited to, thickening keratinous tissue (i.e., building the epidermis and/or dermis and/or subcutaneous layers of the skin and where applicable the keratinous layers of the nail and hair shaft), preventing, retarding, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of, mammalian skin, desquamating, exfoliating, and/or increasing turnover in mammalian skin, reducing the size of pores in mammalian skin, regulating oily/shiny appearance of mammalian skin, preventing, retarding, and/or treating hyperpigmentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin.

Regulating keratinous tissue condition may involve, for example, topically applying to the keratinous tissue a safe and effective amount of a composition, which may be a composition that is modelled according to the exemplary techniques described herein. The amount of the composition that is applied, the frequency of application and the period of use will vary widely depending upon the level of skin care actives and/or other components of a given composition and the level of regulation desired. Such amount, frequency, period of use, and suitable components of the composition, as well as allergic reactions, etc. may be analyzed and/or determined using the modelling techniques described herein, for example.

In some instances, such a composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. A wide range of quantities of the compositions can be employed to provide a skin appearance and/or feel benefit.

Treating keratinous tissue condition can be practiced, for example, by applying a composition in the form of a skin lotion, clear lotion, milky lotion, cream, gel, foam, ointment, paste, emulsion, spray, aerosol, conditioner, tonic, cosmetic, lipstick, foundation, nail polish, after-shave, roll-on or deodorant stick, powder, oil or the like which is intended to be left on the skin or other keratinous tissue for some aesthetic, prophylactic, therapeutic or other benefit (i.e., a “leave-on” composition). After applying the composition to the keratinous tissue (e.g., skin), it may be left on for an appropriate period of time, such as about 15 minutes or several hours (or even longer). Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.). The composition can be dispensed from a bottle, jar, tube, sachet, pouch, container, tottle, vial, ampule, compact, etc. or can be integrally contained within a delivery form such as a wipe. The application of certain compositions may be done using the palms of the hands and/or fingers. The application of certain compositions may be done with the aid of a device or implement such as a cotton ball, swab, pad, brush, eye dropper, puff, sponge, wand, wipe, foam, nonwoven substrate, mask, roll-on applicator, stick applicator, applicator pen, spray applicator, atomizer, razor, etc. In some instances, the application of a topical composition may be performed subsequent to a skin treatment such as cleansing, exfoliation, or tanning.

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, upper lip, and the like). The patch can be occlusive, semi-occlusive or non-occlusive, and can be adhesive or non-adhesive. The composition can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can also contain a source of electrical energy (e.g., a battery) to, for example, increase delivery of the composition and active agents (e.g., iontophoresis). The patch may be left on the keratinous tissue for an appropriate period of time, such as about 5 minutes or overnight as a form of night therapy (or even longer). The modelling techniques described herein may be employed to, for example, model the composition and/or patch or other applicator.

In some instances, other devices can also be employed in conjunction with use of compositions. For example, ultrasound, lasers, heating devices, and the like can be employed to enhance the benefits for skin and hair. The modelling techniques described herein may be employed to model the compositions and/or the effects of the other devices when employed in conjunction with such compositions.

### TEST METHODS FOR EXAMPLES 1-7

### Test Method for Example 1

### Test for Determining Observed Headspace Response Ratio (HRR) Values for AAPD Formulations

Two sets of fabric samples consisting of 32 terry tracers (40×40 cm) each are preconditioned by washing 4 times: 2 times with 70 g Ariel Sensitive (commercial powder detergent nil perfume product from the Procter & Gamble Company) and 2 times without powder at 90° C. One set is designated as a control (nil technology) set and is prepared by washing using a conventional HDL formulation comprising cleaning agents (anionic and nonionic surfactants), solvents, water, stabilizing agents, enzymes, and colorants. The formulation is also spiked with 1% perfume. The second set is prepared by washing using the same HDL formulation containing 1% perfume and Lupasol® WF or HF (polyethyleneamine with a molecular weight of 25,000) supplied by BASF. The fabric samples are washed using Miele Novotronic type W715 washing machines using a short cycle (75 minutes) at 40° C., city water (2.5 mM), no fabric softener added. After the wash the tracers are line dried. When dry, tracers are wrapped in aluminium foil and stored for 5-weeks before analysis using headspace GC/MS analysis.

Headspace GC/MS analysis is carried out by placing about 40 g of fabric in a 1 L closed headspace vessel that is then stored at ambient conditions overnight. After storage, sampling of the headspace is accomplished by drawing a 3 L sample, over 2 hours with a helium flow rate of 25 ml/min, onto the Tenax-TA trap at ambient conditions. The trap is then dry-purged using a reverse-direction helium flow at a rate of 25 ml/min for 30 minutes. In order to desorb trapped compounds, the trap is then heated at 180° C. for 10 minutes directly into the injection-port of a GC/MS. The separation conditions for the GC are a Durawax-4 (60 m, 0.32 mm ID, 0.25 μm Film) column with a temperature program starting at 40° C. and heating to 230° C. at a rate of 4° C./min, holding at 230° C. for 20 minutes. Eluted components are detected using spectrometric detection, and the response is taken as the area of the peak for each perfume component. The results are expressed as the ratio of the areas for a given perfume material of the technology versus nil-technology samples.

### Test Method for Example 2

### Test for Determining Observed Headspace Response Ratio (HRR) Values for AAPD Formulations

Two sets of fabric samples consisting of 32 terry tracers (40×40 cm) each are preconditioned by washing 4 times: 2 times with 70 g Ariel Sensitive (powder nil perfume) and 2 times without powder at 90° C. One set of tracers is designated as a control set (nil technology) and is prepared by washing using an HDL formulation comprising cleaning agents (anionic and nonionic surfactants), solvents, water, stabilizing agents, enzymes, and colorants. The formulation is also spiked with 1% perfume. The second set of tracers is prepared by washing using the same HDL formulation containing 1% perfume and N,N′-Bis-(3-aminopropyl)-ethylenediamine. The fabric samples are washed using Kenmore 80 Series Heavy Duty washing machines using a heavy-duty cycle for 12 minutes at 32° C., 1 mM water, and are then rinsed once at 20° C. using a heavy duty cycle. After the wash the tracers are tumble dried. When dry, tracers are wrapped in aluminium foil and stored for 1-week before analysis using headspace GC/MS analysis.

Headspace GC/MS analysis is carried out according to the procedure listed in Example 1.

### Test Method for Example 3

### Test for Determining Observed Headspace Response Ratio (HRR) Values for PAAPD Formulations

Two sets of fabric samples consisting of 32 terry tracers (40×40 cm) each are preconditioned by washing 4 times: 2 times with 70 g Ariel Sensitive (powder nil perfume) and 2 times without powder at 90° C. One set is designated as a standard (nil technology) set and is prepared by washing using a standard dry-powder formulation containing 1% perfume only. The second set is prepared by washing using a dry-powder formulation containing 1% perfume and Lupasol WF or HF (polyethyleneamine with a molecular weight of 25,000). The fabric samples are washed using Miele Novotronic type W715 washing machines using a short cycle (1 h 15 min) at 40° C., city water (2.5 mM), no fabric softener added. After the wash the tracers are line dried. When dry, tracers are wrapped in aluminium foil and stored for 1-day before analysis using headspace GC/MS analysis. Headspace GC/MS analysis is carried out according to the procedure listed in Example 1.

### Test Method for Example 4

### Modelling Differential Scanning Calorimetric (DSC) Phase-Change Temperatures as a Surrogate Measure of Solubility in Silicone Wash System Solvents

The phase-transition temperatures for all samples are determined using a TA Instruments model Q1000 differential scanning calorimeter with a LNCS accessory under He purge @ 25 mL/min. A sampling interval 0.1 sec/pt is used. The instrument is equilibrated at −160.00° C. The temperature program is started at −160.00° C. for a 2.00 minute hold time, and then the data system is started. The temperature is then increased at a rate of 20.00° C./min to 25.00° C. The temperature is then returned to −160.00° C. at a rate of 20.00° C./min. The temperature is held at −160.00° C. for 2.00 minutes. The sample is reheated to 40.00° C. at a rate of 20.00° C./min. The phase transition temperature is determined from the two heating cycles.

### Test Method for Example 5

### Grass-Stain Removal in Silicone Wash Systems

Fabric samples are cut into 1½×1½ swatches. The fabric samples are then soiled with grass stain using a ½ inch circle template. The soiled fabrics are allowed to dry overnight or a minimum of 3 hours in front of a fan. The swatches are labelled with an ink pen. Test materials are weighed into a glass vial first and are mixed using a vortex mixer. An aliquot of 100 mL of D5 is added to a 16 oz. plastic container with a lid. The test materials are added into the D5, the jar is sealed and the ingredients mixed by manual shaking. Two marbles are added to the container to aid in agitation. The soiled swatches are placed into the D5 cleaning solutions. The lids are secured and the containers are placed onto a Lab-Line model 3689 multi-wrist shaker. The samples are shaken at highest speed for 30 minutes. After 30 minutes, the swatches are removed from the solution and squeezed lightly to remove excess solution. The swatches are dried flat on drying screens over night in a chemical fume hood, or are dried in a clothes dryer. When swatches are dried, they are graded by SRI grading on the Image Analyses system. The stain-removal index (SRI) values are determined using the Laundry Image Analysis system. Color differences are measured by comparing the color of the unstained fabric to the color of the stained fabric before and after cleaning. Color difference, also known as delta-E (or delta-Lab), is quantified as the distance in CIE Color Space between observed CIE Lab values for stained and unstained fabric. The SRI is computed as follows:
SRI=(AB)−(AD)/AB
Where, AB represents the delta-Lab value comparing the unstained and stained fabric before washing, and AD is the delta-Lab value comparing the unstained fabric colour before wash to the stained fabric color after wash.

### Test Method for Example 6

### Perfume/LDL (Liquid Dish) Formulation Color Stability

Test samples are prepared by adding 0.02% of perfume raw material to base liquid detergent product consisting cleaning and sudsing agents (anionic and nonionic surfactants) dispensing aid (ethyl alcohol), water, stabilizing agents, protease enzyme, and colorant. The samples are mixed well by manually shaking. They are then subjected to a rapid aging test consisting of storage, in the dark, at 50° C. for 10 days. The samples are then compared to an aged blank (nil perfume) sample using a Hunter Colorquest-II spectrophotometer, or equivalent. The color difference between the aged and control sample is quantified by determining the delta-Lab value, defined as the distance in CIE Color Space between observed CIE Lab values for the control and aged samples.

### Test Methods for Example 7

### Ester-type Perfume Raw Material Hydrolysis by Lipase Enzymes

Analytical Test: Perfume ester stability is assayed in the following manner. Blends of perfume esters disclosed in the present application are made by ad-mixing perfume ester raw materials that are disclosed in the present application in equal weight percents. The resultant perfume is added at a 0.3% level to a liquid detergent, sold under the trade name TIDE®. Ester degradation is monitored at time 0 and 24 hours after storage at 20° C. to 25° C., both in-product and in a wash solution made by adding 1.5 g of the above liquid detergent to 1 liter of water. The ester content of the resultant liquids and solutions is assayed via standard headspace gas chromatographic methods as described, for example, in Janusz Pawliszyn “Application of Solid Phase Microextraction”, RS.C, Chapter 26, pages 349-457, 1999. Specifically headspace solid phase microextraction (SPME) is followed by thermal desorption GC/MS analysis. The SPME fiber is coated with 100% polydimethyl siloxane (PDMS). The thickness of the polymer film on the fiber is 100-um. Samples are put into 20-mL headspace vials with septum seal, and equilibrated for 60 minutes before analysis. For sampling, the fiber is placed in the headspace of the sample vial and absorption is carried out for 20 minutes. Then the samples are injected to the GC column under 240° C. for 5 minutes in the injector. GC/MS system used for this work is a 5973 MS couple with 6890 GC, both from Agilent technologies. Separation of the PRM components is accomplished using a 60-m×250-um i.d. capillary column coated with 1-um PDMS phase.

Beaker Test: A test solution is prepared by adding 500 mL tap water at about 20° C. to a plastic beaker with loose lid. Next, 5 g of Perfumed Ariel Regular Endeavour is added and manually stirred, and then placed on magnetic stirrers and graded by a trained perfumer at 1, 3, 5, 10, and 15 minute intervals vs. a nil Lipex sample prepared in the same way. Possible grades assigned to samples include: no change, slight change, moderate change, and significant change.

### EXAMPLES

### Example 1

### Amine-Assisted Perfume Delivery (AAPD)

The structures of the following perfume raw materials (PRMs) are entered into a Sybyl® database by sketching or by importing the structures from a compatible file format: hexyl cinnamic aldehyde; 2,6-dimethyl-5-heptenal; 2-methyl-undecanal; n-decanal; n-undecanal; n-dodecanal; 4-isopropyl benzaldehyde; amyl cinnamic aldehyde; n-nonanal; 3-(3-isopropylphenyl)butyraldehyde; 2-heptylcyclopentanone; 2,6,10-trimethyl-undec-9-enal; p-tert-butylhydrocinnamic aldehyde; 7-methoxy-6,7-dihydrocitronellal; 4-(4-Methyl-3-penten-1-yl)-3-cyclohexen-1-carboxaldehyde; 3,7-dimethyloctan-1-al; 6-methoxy-2,6-dimethylheptan-1-al; (R)-2-methyl-5-(1-methylethenyl)-2-cyclohexen-1-one; trans-4-decen-1-al; 2-Butenoic acid, 1-cyclohexylethyl ester; gamma-methyl ionone; 2-(2-(4-methyl-3-cyclohexen-1-yl)propyl)cyclopentanone; alpha-methyl ionone; benzaldehyde; benzyl acetate; camphor; cis-3-hexenyl acetate; 3,7-dimethyl-6-octenonitrile; n-dodecanonitrile; 2-buten-1-one, 1-(2,6,6-trimethyl-3-cyclohexen-1-yl)-; 7-octen-2-ol, 2,6-dimethyl-, (±)-; 1-oxacyclohexadecan-2-one; 4,7-methano-1H-inden-6-ol, 3a,4,5,6,7,7a-hexahydro-8,8-dimethyl-, acetate; 2,4-dimethyl-3-cyclohexene-1-carboxaldehyde; 2-(4-tert-Butylbenzyl)propionaldehyde; (±)-3,7-dimethyl-1,6-octadien-3-ol; 1,6-octadien-3-ol, 3,7-dimethyl-, acetate; ethanone, 1-(2,3,4,7,8,8a-hexahydro-3,6,8,8-tetramethyl-1H-3a,7-methanoazulen-5-yl)-, [3R-(3.alpha., 3a.beta., 7.beta., 8a.alpha.)]-; 3,7-dimethyloctan-3-ol; 2-tert.butylcyclohexyl acetate. Initial 3D atomic coordinates for each structure are computed using Concord®. The structures are exported to Spartan using a Sybyl® MOL2 format file. A conformational search is performed using molecular mechanics (MMFF force field) to identify the lowest-energy conformer for each structure. The energy of the structures is further optimized using quantum mechanics (PM3). The structures are exported into a new Sybyl® database using a Sybyl® MOL2 format file. Partial atomic charges are computed using the Gasteiger-Huckel method, as found in Sybyl®, without further structure optimization. The structures are exported to ADAPT using a Sybyl® MOL file format, including the partial atomic charges. Using ADAPT, the desired set of molecular descriptors is computed. The observed headspace response ratio (HRR) values for AAPD formulations are collected according to the test method for this example. The log (logarithm, base 10) of the observed HRR is computed and is used as the dependent property. This response is corrected for differences in the molecular weights of the PRMs as needed. The dependent property values, and other independent variables as needed, are imported into ADAPT. Objective feature selection is performed (as described in “J. Chem. Inf. Comp. Sci, 2000, 40,” 81-90). Descriptor selection is performed using a genetic algorithm, simulated annealing, or both. The model is recomputed using multiple linear regression analysis and the diagnostic statistics are evaluated. The appropriate descriptors are exported to Minitab further model verification and diagnostic tests are performed. The model can be further validated using an external prediction set. The following model is generated by the aforementioned process:
Log(HRR)=0.710−0.0414×(WNHS-1)+0.762×(RNH)−0.0217×(RNCS)+2.18×(FNHS-1)+0.0772×(3SP3)−0.011×(ALLP-3)
where: WNHS-1 is the type-1 weighed negative hydrophobic surface, RNH is the relative negative hydrophobicity, FNHS-1 is the type-1 fractional negative hydrophobic surface computed as described in “J. Chem. Inf. Comput. Sci. 2004, 44,” 1010-1023. RNCS is the relative negative charged surface computed as described in “Anal. Chem. 1990, 62,” 2323-2329. 3SP3 is a simple count of occurrences of sp³-hybridized carbon atoms attached to exactly three other carbon atoms. ALLP-3 is the total weighted number of paths in the range of lengths from 1 to 46 computed as described in “Comp. Chem., 1979, 3,” 5-13.

The model is applied and predicts that the following PRMS are useful in AAPD: benzophenone; 1-methyl-4-(1-methylethyl)-7-oxabicyclo[2.2.1]heptane; 1,3,3-trimethyl-2-oxabicyclo[2.2.2]octane; 1-(2,6,6-trimethyl-2-cyclohexen-1-yl)-2-Buten-1-one; 1-methoxy-4-(2-propenyl)-benzene; (1R,2S,5R)-5-methyl-2-(1-methylethenyl)-cyclohexanol; 4-methyl acetophenone; 3,7-Dimethyl-1,6-octadien-3-yl isobutanoate; (1R,4S,4aS,6R,8aS)-octahydro-4,8a,9,9-tetramethyl-1,6-methanonaphthalen-1(2H)-ol; 6-methyl-8-(1-methylethyl)-bicyclo[2.2.2]oct-5-ene-2-carboxaldehyde.

### Example 2

### Amine-Assisted Perfume Delivery (AAPD)

The structures of the following perfume raw materials (PRMs) are entered into a Sybyl® database by sketching or by importing the structures from a compatible file format: hexyl cinnamic aldehyde; 2,6-dimethyl-5-heptenal; 2-methyl-undecanal; n-decanal; n-dodecanal; 4-isopropyl benzaldehyde; amyl cinnamic aldehyde; oxacycloheptadec-8-en-2-one, (Z)-; n-octanal; n-nonanal; 3-(3-isopropylphenyl)butyraldehyde; 2-heptylcyclopentanone; 2,6,10-trimethyl-undec-9-enal; (E)-1-(2,6,6-trimethyl-1,3-cyclohexadien-1-yl)-2-buten-1-one; 2-buten-1-one, I-(2,6,6-trimethyl-2-cyclohexen-1-yl)-, (E)-; oxacyclohexadecen-2-one; 3,7-dimethyloctan-1-al; 6-methoxy-2,6-dimethylheptan-1-al; benzoic acid, 2-hydroxy-, hexyl ester; benzenepropanal, .alpha.-methyl-4-(2-methylpropyl)-; alpha,alpha-dimethyl-p-ethylphenylpropanal; 3-(4-methylcyclohex-3-en-1-yl)-butyraldehyde; [3aR-(3aa,5ab,9aa,9bb)]-dodecahydro-3a,6,6,9a-tetramethylnaphtho[2,1-b]furan; 2-butenoic acid, 1-cyclohexylethyl ester; gamma-methyl ionone; 3-Buten-2-one, 4-(2,6,6-trimethyl-1-cyclohexen-1-yl)-, (3E)-; 2-(2(4-methyl-3-cyclohexen-1-yl)propyl)-cyclopentanone; 2-propenyl hexanoate; 1-penten-3-one, 1-(2,6,6-trimethyl-2-cyclohexen-1-yl)-, [R-(E)]-; 4-methoxy-benzaldehyde; benzaldehyde; benzyl acetate; camphor; 3,7-dimethyl-6-octenonitrile; n-dodecanonitrile; cyclohexene, 1-methyl-4-(1-methylethenyl)-, (4R)-; 2-buten-1-one, 1-(2,6,6-trimethyl-3-cyclohexen-1-yl)-; 7-octen-2-ol, 2,6-dimethyl-, (±)-; butanoic acid, 2-methyl-, ethyl ester, (±)-; 1-oxacyclohexadecan-2-one; 2,4-dimethyl-3-cyclohexene-1-carboxaldehyde; p-tert.butyl-alpha-methyldihydrocinnamic aldehyde; (±)-3,7-dimethyl-1,6-octadien-3-ol; 1,6-octadien-3-ol, 3,7-dimethyl-, acetate; methyl 2-aminobenzoate; ethanone, 1-(2,3,4,7,8,8a-hexahydro-3,6,8,8-tetramethyl-1H-3a,7-methanoazulen-5-yl)-, [3R-(3.alpha., 3a.beta., 7.beta., 8a.alpha.)]-; 2H-pyran, tetrahydro-4-methyl-2-(2-methyl-1-propenyl)-, (2R,4R)-; 3,7-dimethyloctan-3-ol; 4-methyl-3-decen-5-ol; 2-tert.butylcyclohexyl acetate. Initial 3D atomic coordinates for each structure are computed using Concord®. The structures are exported to Spartan using a Sybyl® MOL2 format file. A conformational search is performed using molecular mechanics (MMFF force field) to identify the lowest-energy conformer for each structure. The energy of the structures is further optimized using quantum mechanics (PM3). The structures are exported into a new Sybyl® database using a Sybyl® MOL2 format file. Partial atomic charges are computed using the Gasteiger-Huckel method, as found in Sybyl®, without further structure optimization. The structures are exported to ADAPT using a Sybyl® MOL file format, including the partial atomic charges. Using ADAPT, the desired set of molecular descriptors is computed. The observed headspace response ratio (HRR) values for AAPD formulations are collected according to the test method for this example. The other model steps, as described in Example 1, are applied. The following model is generated:
Log(HRR)=0.413−0.0406×(RNHS)+0.0165×(SSAH)−0.287×(3SP3)+0.260×(GEOH-3)−0.0913×(KAPA-5)+0.000342×(PPHS-2)
where: RNHS is the relative negative hydrophobic surface and PPHS-2 is the type-2 partial positive hydrophobic surface computed as described in “J. Chem. Inf. Comput. Sci. 2004, 44,” 1010-1023. SSAH is the sum of the solvent-accessible surface area of hydrogen atoms that can participate in hydrogen-bond formation computed as described in “J. Chem. Inf. Comput. Sci. 1992, 32,” 306-316. 3SP3 is a simple count of occurrences of sp³-hybridized carbon atoms attached to exactly three other carbon atoms. GEOH-3 is the third major molecular axis independent of mass (e.g. “thickness”). KAPA-5 is type-2 Kier alpha-modified shape descriptor computed as described in “Quant. Struct.-Act. Relat., 1986, 5,” 7-12. The model is applied and predicts that the following PRMS are useful in AAPD: 3,7-dimethyl-1,6-octadien-3-yl octanoate; 2,2,5-trimethyl-5-pentyl-cyclopentanone; 4-(1,5-dimethyl-4-hexenylidene)-1-methyl-cyclohexene; benzoic acid, 2-[[3-[4-(1,1-dimethylethyl)phenyl]-2-methylpropylidene]amino]-, methyl ester; 1-(5,6,7,8-tetrahydro-3,5,5,6,8,8-hexamethyl-2-naphthalenyl)-ethanone; 2,6-dimethyl-5,7-octadien-2-ol; 1-(2,3-dihydro-1,1,2,3,3,6-hexamethyl-1H-inden-5-yl)-ethanone; 4-(3-Methoxy-4-hydroxyphenyl)-2-butanone; 1-Ethoxy-1-(phenylethoxy)ethane; 1-Methyl-4-isopropenyl-6-cyclohexen-2-ol.

### Example 3

### Polymer Amine-Assisted Perfume Delivery (PAAPD)

The structures of the following perfume raw materials (PRMs) are entered into a Sybyl® database by sketching or by importing the structures from a compatible file format: 3-Buten-2-one, 4-(2,6,6-trimethyl-1-cyclohexen-1-yl)-, (3E)-; 2-Cyclohexen-1-one, 2-methyl-5-(1-methylethenyl)-, (R)-; 2-Butenoic acid, 1-cyclohexylethyl ester; n-decyl aldehyde; 1-(2,6,6-Trimethyl-3-cyclohexen-1-yl)-but-2-en-1-one; 3-Cyclohexene-1-carboxaldehyde, 4-(4-methyl-3-pentenyl)-; Benzenepropanal, 4-ethyl-.alpha., .alpha.-dimethyl-; Benzenepropanal, .beta.-methyl-3-(1-methylethyl)-; 3-Buten-2-one, 4-(2,2-dimethyl-6-methylenecyclohexyl)-3-methyl-; 5-Cyclohexadecen-1-one; Octanal, 2-(phenylmethylene)-; 3,4,5,6,6-Pentamethylhept-3-en-2-one; 2-(4-tert-Butylbenzyl)propionaldehyde; 2,6-Dimethylhept-5-enal; Octanal, 7-methoxy-3,7-dimethyl-; 3-Cyclohexene-1-carboxaldehyde, 2,4-dimethyl-. Initial 3D atomic coordinates for each structure are computed using Concord®. The structures are exported to Spartan using a Sybyl® MOL2 format file. A conformational search is performed using molecular mechanics (MMFF force field) to identify the lowest-energy conformer for each structure. The energy of the structures is further optimized using quantum mechanics (PM3). The structures are exported into a new Sybyl® database using a Sybyl® MOL2 format file. Partial atomic charges are computed using the Gasteiger-Huckel method, as found in Sybyl®, without further structure optimization. The structures are exported to ADAPT using a Sybyl® MOL file format, including the partial atomic charges. Using ADAPT, the desired set of molecular descriptors is computed. The observed headspace response ratio (HRR) values for PAAPD formulations are collected according to the test method for this example. The other model steps, as described in Example 1, are applied. The following model is generated:
Log(HRR)=7.84−0.0120×(PPSA-2)−0.117×(RNCS)-10.2×(RPCG)
where: PPSA-2 is the type-2 partial positive surface areas descriptor, RNCS is the relative negative charged surface, and RPCG is the relative positive charge, all computed as described in “Anal. Chem. 1990, 62,” 2323-2329. The model is applied and predicts that the following PRMS are useful in PAAPD: 2-Phenylpropionaldehyde; camphor; 4-isopropyl benzaldehyde; 2-Methyl-3-tolylpropionaldehyde;
4-(1-methylethenyl)-1-cyclohexene-1-carboxaldehyde; 4-(1,1-dimethylpropyl)-cyclohexanone; 2-pentylcyclopentanone; 4-(2,5,6,6-Tetramethyl-2-cyclohexen-1-yl)-3-buten-2-one; 3,7-Dimethyl-2,6-octadienal; 3-(3,4-Methylenedioxyphenyl)-2-methylpropanal.

### Example 4

### Modeling Differential Scanning Calorimetric (DSC) Phase-Change Temperatures as a Surrogate Measure of Solubility in Silicone Wash System Solvents

The structures of the following test materials are entered into a Sybyl® database by sketching or by importing the structures from a compatible file format: 2-(2-(3-oxo-3-(pentan-3-yloxy)propoxy)ethoxy)ethyl 2-ethylbutanoate: 2-(2-(2-(2-hydroxyethoxy)ethoxy)ethoxy)ethyl stearate; 2-(2-butoxyethoxy)ethanol; 2-(2-(hexyloxy)ethoxy)ethanol; 1-(2-(2-butoxyethoxy)ethoxy)butane; 2-(2-hydroxyethoxy)ethyl dodecanoate; 2-(2-(2-butoxyethoxy)ethoxy)ethanol; 3,6,9,12,15-pentaoxapentacosan-1-ol; 3,6,9,12,15,18-hexaoxaoctacosan-1-ol; 3,6,9,12,15,18,21,24,27-nonaoxaheptatriacontan-1-ol; 2-(2-(2-(tetradecyloxy)ethoxy)ethoxy)ethanol; 3,6,9,12,15,18,21-heptaoxapentatriacontan-1-ol; 3,6,9,12-tetraoxaheptacosan-1-ol; 3,6,9,12,15-pentaoxatriacontan-1-ol; 3,6,9,12,15,18,21-heptaoxahexatriacontan-1-ol; heptane-1,2-diol; octan-1-ol; 2-ethylhexan-1-ol; 2-ethylhexan-1-amine; 2-(hexylamino)ethanol; 4-nonylphenol; (E)-2-(2-(octadec-9-enyloxy)ethoxy)ethanol; 3-(octadecyloxy)propane-1,2-diol; butan-1-amine; (9Z,12Z)-octadeca-9,12-dien-1-ol; 2-(4-(2,4,4-trimethylpentan-2-yl)phenoxy)ethanol; 2-(4-nonylphenoxy)ethanol; (9Z, 12Z)-octadeca-9,12-dienoic acid; 2-(tridecyloxy)ethanol; 3,6,9,12,15-pentaoxaoctacosan-1-ol; nonanoic acid; (Z)-octadec-9-en-1-ol; (Z)-octadec-9-en-1-amine; (16S, 19S)-16,19-diisobutyl-16,19-dimethyl-3,6,9,12,15,20,23,26,29,32-decaoxatetratriacont-17-yne-1,34-diol; 22-hexyl-3,6,9,12,15,18,21-heptaoxatriacontan-1-ol; heptanoic acid; (E)-hexadec-9-enoic acid; nonanoic acid; (E)-octadec-9-enoic acid; (E)-octadec-9-enoic acid; 2-methyldodecanoic acid; (E)-icos-9-enoic acid; undec-10-enoic acid; undecanoic acid; cyclohexanecarboxylic acid; (E)-docos-13-enoic acid; dodecanoic acid; (E)-octadec-8-enoic acid; tridecanoic acid; tetradecanoic acid; palmitic acid; 11-hydroxyundecanoic acid; 13-(cyclopent-2-enyl)tridecanoic acid; nonadecanoic acid; 2-hydroxyoctanoic acid; icosanoic acid; docosanoic acid; tetracosanoic acid; hexacosanoic acid; 9,10-dihydroxyoctadecanoic acid; triacontanoic acid; 9,10,16-trihydroxyhexadecanoic acid; 2-octylmalonic acid; icosanedioic acid; tetradecanedioic acid; dodecanedioic acid; decanedioic acid; octanedioic acid; triethanolamine; decyl bis(2-hydroxyethyl)carbamate; 2-(1,3-dihydroxy-2-(hydroxymethyl)propan-2-ylamino)acetic acid; 2-hydroxyoctanoic acid; 5,6,7,8-tetramethyldec-9-yne-3,4-diol; 2-hydroxyhexanoic acid. Initial 3D atomic coordinates for each structure are computed using Concord®. Gasteiger-Huckel partial atomic charges are computed for each structure. The initial 3D conformations are optimized using the Tripos force field, including electrostatic terms. The optimized 3D conformers are exported with the corresponding partial atomic charge data. This data is stored in an ADAPT database. The observed DSC temperatures in units of degrees Kelvin [DSC(K)] are collected, according to the test method for this example, and added to the ADAPT database. The other model steps, as described in Example 1, are applied. The following model is generated
DSC(K)=184.4+625.6×(RSAM)+109.6×(V6P)+17.78×(CNTH)−1.991×(DPSA-3)-223.4×(FNSA-2)−0.9079×(GEOH-6)
where: RSAM is the ratio of the solvent-accessible surface area of hydrogen-bond acceptor groups to the total solvent-accessible surface area of the molecule, and CNTH is the simple count of hydrogen-bond donor groups, both computed as described in “J. Chem. Inf. Comput. Sci. 1992, 32,” 306-316. V6P is the sixth-order valence-corrected path molecular connectivity index, DPSA-3 is the type-3 difference charge-partial surface area descriptor and FNSA-2 is the type-2 fractional charged partial surface-area descriptor, both computed as described in “Anal. Chem. 1990, 62,” 2323-2329. GEOH-6 is the ratio of the lengths of the second and third major geometric axes of the structure. The model can be applied to predict the suitability of materials for use in silicone wash system solvents, for example, D5 (Decamethylcyclopentasiloxane).

### Example 5

### Grass-Stain Removal in Silicone Wash Systems

The structures of the following test materials are entered into a Sybyl® database by sketching or by importing the structures from a compatible file format: 2,4,7,9-tetramethyldecane-4,7-diol; oleic acid; 2-butyl-N,N-bis(2-hydroxyethyl)octanamide; 2,2′-(3-(2-ethylhexyloxy)-2-hydroxypropylazanediyl)diethanol; 2-butyl-N,N-bis(2-hydroxypropyl)octanamide; 2,2′-(2-hydroxytetradecylazanediyl)diethanol; 2-(2-(2-(2,6,8-trimethylnonan-4-yloxy)ethoxy)ethoxy)ethanol; 2-(2-(2-(tridecan-6-yloxy)ethoxy)ethoxy)ethanol; 2-(3,4-dihydroxytetrahydrofuran-2-yl)-2-hydroxyethyl dodecanoate; (Z)-2-(2-(2-(octadec-9-enyloxy)ethoxy)ethoxy)acetic acid; 16-pentyl-3,6,9,12,15-pentaoxatricosan-1-ol; 7,10,13,17-tetraethyl-3,6,9,12,15-pentaoxahenicosan-1-ol; 7,10,13,17,20,23-hexaoxanonacosan-15-ol; 3,3′-(3-hydroxypropylazanediyl)bis(1-(2-ethylhexyloxy)propan-2-ol); 19-isobutyl-21,23-dimethyl-3,6,9,12,15,18-hexaoxatetracosan-1-ol; 10,13,16,20-tetraethyl-3,6,9,12,15,18-hexaoxatetracosan-1-ol; 2-(3,6,9,12,15,18-hexaoxaoctacosyloxy)tetrahydro-2H-pyran; 22-pentyl-3,6,9,12,15,18,21-heptaoxanonacosan-1-ol; 2-(2-(3,4-bis(2-hydroxyethoxy)tetrahydrofuran-2-yl)-2-(2-hydroxyethoxy)ethoxy)ethyl dodecanoate; (Z)-3,6,9,12,15,18-hexaoxahexatriacont-27-en-1-oic acid; 2-(3,6,9,12,15,18,21,24-octaoxatetratriacontyloxy)tetrahydro-2H-pyran; 28-pentyl-3,6,9,12,15,18,21,24,27-nonaoxapentatriacontan-1-ol; 9,12,15,18,21,24,27,30,33-nonaoxatritetracontane; 2-(5,8,11,14,17,20,23,26-octaoxaheptatriacontan-3-yloxy)tetrahydro-2H-pyran; 28-pentyl-3,6,9,12,15,18,21,24,27-nonaoxapentatriacontyl acetate; 2-(6-ethyl-5,8,11,14,17,20,23,26,29-nonaoxatetracontan-3-yloxy)tetrahydro-2H-pyran; 37-pentyl-3,6,9,12,15,18,21,24,27,30,33,36-dodecaoxatetratetracontan-1-ol; 6,9-diethyl-5,8,11,14,17,20,23,26,29,32,35,38,41,44,47,50-hexadecaoxahexacontan-3-ol; 8-pentyl-9,12,15,18,21,24,27,30,33,36,39,42,45,49-tetradecaoxadohexacontan-47-ol; 6,9,12,15,18,21,24-heptaethyl-5,8,11,14,17,20,23,26,29,32,35,38,41,44,47,50-hexadecaoxahexacontan-3-ol; 6,9,12,15,18-pentaethyl-5,8,11,14,17,20,23,26,29,32,35,38,41,44,47,50,53,56,59-nonadecaoxanonahexacontan-3-ol; 17-(3,4-bis(14-hydroxy-3,6,9,12-tetraoxatetradecyloxy)tetrahydrofuran-2-yl)-32-hydroxy-3,6,9,12,15,18,21,24,27,30-decaoxadotriacontyl oleate; (Z)-((2S,3S,4S,5R)-2-((2R,3R,4S,5S,6R)-4,5-dihydroxy-3-(oleoyloxy)-6-(oleoyloxymethyl)tetrahydro-2H-pyran-2-yloxy)-3,4-dihydroxytetrahydrofuran-2,5-diyl)bis(methylene) dioleate. The structures are exported using an SDF (structure-data file) format. The series of topological descriptors available in MolconnZ™ are computed for these structures and stored in an ADAPT database. The observed Stain Removal Index (SRI) is collected for the test materials according to the test method for this example. The observed SRI values are adjusted to account for differences in the molecular weight of the test materials.
SRI_{mw}═SRI_{obs}×MWₜₑₛₜ/MWₘᵢₙ
where SRI_{mw} is the molecular-weight adjusted SRI, SRI_{obs} is the original observed SRI, MWₜₑₛₜ is the molecular weight of the test material, and MWₘᵢₙ is the minimum molecular weight observed for the whole set of test materials. SRI_{mw} is used as the dependent property for model development. The SRI_{mw} values are imported into ADAPT. The other model steps, as described in Example 1, are applied. The following model is generated:
SRI_{mw}=−44.53+76.98×(nrings)−71.90×(dxp4)+10.08×(SsCH3)+3.911×(SssCH2)+1.340×(SHBint6)
where: nrings is a simple count of rings in the structure, dxp4 is the difference forth-order path molecular connectivity index, SsCH3 is the sum of the electrotopological-state indices for methyl groups, SssCH2 is the sum of the electrotopological-state indices for methylene groups, and SHBint6 is the sum of the electrotopological-state indices for groups that can participate in an intramolecular hydrogen-bond that are separated by a path of 6 (six bonds).

The model can be applied to predict the suitability of materials for grass stain removal in silicone wash system solvents, for example, D5 (Decamethylcyclopentasiloxane).

### Example 6

### Perfume/LDL (Liquid Dish) Formulation Color Stability

The structures of the following perfume raw materials (PRMs) are entered into a Sybyl® database by sketching or by importing the structures from a compatible file format: trans-4-Decen-1-al; alpha-terpineol; 1-(2,3,4,7,8,8a-hexahydro-3,6,8,8-tetramethyl-1H-3a,7-methanoazulen-5-yl)-[3R-(3.alpha., 3a.beta., 7.beta., 8a.alpha.)]-ethanone; n-decanal; 10-Undecenal; n-Lauraldehyde; n-Hexanal; n-Octanal; n-Nonanal; Acetic acid, (3-methylbutoxy)-, 2-propenyl ester; Allyl amyl glycolate; Allyl caproate; Allyl heptanoate; alpha-Damascone; alpha-lonone; alpha-Terpinyl acetate; 5-Cyclohexadecen-1-one; Amyl acetate; Benzaldehyde; Benzyl acetate; Benzyl salicylate; beta-Damascone; beta-lonone; Butyl acetate; cis-3-Hexenyl acetate; 2-hexyl-2-Cyclopenten-1-one; (S)-3,7-dimethyl-6-octenal; 3,7-Dimethyl-6-octen-1-yl acetate; 3,7-Dimethyl-6-octenonitrile; 3a,4,5,6,7,7a-hexahydro-4,7-methano-1H-inden-5-yl isobutyrate; alpha-Methyl-p-isopropylhydrocinnamaldehyde; 4,7-Methano-1H-inden-6-ol, 3a,4,5,6,7,7a-hexahydro-, propanoate; Octahydro(2H) 1-benzopyran-2-one; 1-(2,6,6-trimethyl-1,3-cyclohexadien-1-yl)-2-Buten-1-one; delta-damascone; Dibenzyl ether; Diethyl malonate; Dihydromyrcenol; Dihydroisojasmonate; Dimethylbenzylcarbinyl acetate; Dimethylbenzylcarbinyl butyrate; Ethyl-2-methylbutyrate; ethyl butyrate; Ethyl caproate; Ethyl methyl phenyl glycidate; 2-methoxy-4-(1-propenyl)-phenol; gamma-Decalactone; gamma-Nonalactone; gamma-Undecalactone; Cyclopentaneacetic acid, 3-oxo-2-pentyl-, methyl ester; 2-Methyl-3-(3,4-methylenedioxyphenyl)-propanal; 1,3-Benzodioxole-5-carboxaldehyde; Hexyl acetate; Hexylcinnamic aldehyde; Hexyl salicylate; 7-hydroxy-3,7-dimethyl-octanal; 4,4a,5,9b-tetrahydroindeno[1,2-d]-1,3-dioxin; Isobornyl acetate; 4-(4-hydroxy-4-methylpentyl)cyclohex-3-enecarbaldehyde; 2-Ethyl-4-(2,2,3-trimethylcyclopent-3-enyl-1)-2-buten-1-ol; 2-(4-tert-Butylbenzyl)propionaldehyde; Linalyl acetate; (Z)-3-Dodecenal; Ethyl-2-methyl pentanoate; 1,3-Dioxolane-2-acetic acid, 2-methyl-, ethyl ester; gamma-methyl ionone; 1,4-Dioxacycloheptadecane-5,17-dione; 1-(1,2,3,4,5,6,7,8-octahydro-2,3,8,8-tetramethyl-2-naphthalenyl)-ethanone; Phenoxyethyl isobutyrate; 3-methyl-2-butenyl acetate; 2,4-dimethyl-4-phenyltetrahydrofuran; 3-(4-Isobutyl-phenyl)-2-methyl-propionaldehyde; trans-3,7-Dimethyl-2,6-octadien-1-ol; 2,4-Dimethyl-3-Cyclohexene-1-carboxaldehyde; 4-hydroxy-3-methoxy-benzaldehyde; 3-hydroxy-2-methyl-4H-Pyran-4-one; Cyclohexanol, 2-(1,1-dimethylethyl)-, acetate; cis-4-(1,1-dimethylethyl)-cyclohexanol acetate. Initial 3D atomic coordinates for each structure are computed using Concord®. Gasteiger-Huckel partial atomic charges are computed for each structure. The initial 3D conformations are optimized using the Tripos force field, including electrostatic terms. The 3D coordinates are exported to Spartan and a conformational search for the lowest-energy conformer is performed using molecular mechanics optimizations, and the MMFF force field. The conformations are further optimized using semi-empirical methods (PM3). The semi-empirical optimized structures, including the Mulliken partial atomic charges, are exported and stored in a new Sybyl® database. The HOMO and LUMO level energy values and also the Bandgap values computed in Spartan using the PM3 method are exported as a text-file. The structures and corresponding Mulliken partial atomic charge data are exported from Sybyl® stored in an ADAPT database. The HOMO, LUMO, and Bandgap are also stored in the ADAPT database. The rest of the desired set of molecular descriptors is computed in ADAPT. The observed color stability data for the test materials are collected according to the test method for this example. The observed delta-Lab values are adjusted to account for differences in molecular weight of the perfume raw materials by multiplying the observed delta-Lab by the ratio of the molecular weight of the test perfume raw material and the minimum molecular weight observed for any of the test perfume raw materials.
deltaLab_{mw}=deltaLab_{obs}×MWₜₑₛₜ/MWₘᵢₙ
The logarithm (base-10) of the reciprocal of the molecular-weight adjusted delta-Lab value (i.e., log(1/deltaLab_{mw}), is computed and used as the dependent property in the subsequent model development step. These values are added to the ADAPT database. The other model steps, as described in Example 1, are applied. The following model is generated:

deltaLab_{mw}=−3.26−0.0161×(GEOH-4)+0.280×(Egap)+0.388×(RPHS)+10.7×(FNHS-3)+0.165×(3SP2)−5.94×(FNSA-3)−0.0193×(RNCS) where: GEOH-4 is the ratio of the lengths of the first and second principal geometric axes of the structure. Egap is the HOMO-LUMO energy gap computed in Spartan. RPHS is the relative positive hydrophobic surface area of the structure and FNHS-3 is the type-3 fractional hydrophobic surface area of the structure, both computed as described in “J. Chem. Inf. Comput. Sci. 2004, 44,” 1010-1023. 3SP2 is the simple count of occurrences of a sp²-hybridized carbon bonded to three and only three other carbons. FNSA-3 is the type-3 fractional charged surface area of the structure, and RNCS is the relative negative charged surface area of the structure, both computed as described in “Anal. Chem. 1990, 62,” 2323-2329.

The model is applied and predicts that the following PRMs are useful in liquid dish formulations: cyclooct-4-en-1-yl methyl carbonate; 1H-3a,7-mMethanoazulen-6-ol, octahydro-3,6,8,8-tetramethyl-, (3R,3aS,6R,7R,8aS)-; 7-methyl-3-methylene-1,6-octadiene; 1-(2,3-dihydro-1,1,2,3,3,6-hexamethyl-1H-inden-5-yl)-ethanone; (E)-isobutyric acid, 3,7-dimethyl-2,6-octadienyl ester; 1,3,3-trimethyl-2-norbornanyl acetate; 2-methyl-5-(1-methylethenyl)-2-cyclohexen-1-one; 1-oxacyclohexadecan-2-one; ethyl hexanoate; 3-(3-Isopropyl-phenyl)-butyraldehyde.

### Example 7

### Ester-Type Perfume Raw Material Hydrolysis by Lipase Enzymes

The structures of the following test ester-type PRMs are entered into a Sybyl® database by sketching or by importing the structures from a compatible file format: allyl amyl glycolate; allyl caproate; allyl cyclohexyl propionate; amyl salicylate; benzyl acetate; benzyl salicylate; cis-neryl butyrate; citronellyl acetate; cyclohexyl salicylate; dimethyl benzyl carbinyl acetate; ethyl 2-methyl pentanoate; ethyl butyrate; ethyl-2-methyl butyrate; 3a,4,5,6,7,7a-hexahydro-8,8-dimethyl-4,7-Methano-1H-inden-6-ol, acetate; (3aR,4S,7R,7aR)-rel-octahydro-4,7-Methano-3aH-indene-3a-carboxylic acid, ethyl ester; 3a,4,5,6,7,7a-hexahydro-8,8-dimethyl-4,7-Methano-1H-inden-6-ol, propanoate; gamma-dodecalactone; hexyl acetate; hexyl salicylate; isopropyl-2-methyl butyrate; linalyl acetate; methyl dihydrojasmonate; methyl phenyl carbinyl acetate; ortho-t-butyl cyclohexyl acetate; trans-geranyl acetate; trans-geranyl butyrate; cis-4-(1,1-dimethylethyl)-cyclohexanol acetate. Initial 3D atomic coordinates for each structure are computed using Concord®. Gasteiger-Huckel partial atomic charges are computed for each structure. The initial 3D conformations are optimized using the Tripos force field, including electrostatic terms. The optimized 3D conformers with the corresponding partial atomic charge data are exported and stored in an ADAPT database. The desired set of molecular descriptors are computed using ADAPT. The descriptor values are exported to a text file. The observed perfume/lipase hydrolysis data are collected, according to a test method for this example. With respect to the analytical test, perfume raw materials are designated as stable if they show 30% or less hydrolysis during the testing process, and are designated as unstable if more than 30% hydrolysis is observed. If a beaker test is used, perfume materials that exhibit no change or a slight change are classified as stable, and those perfume materials that exhibit a moderate or a significant change are classified as unstable. The descriptor data and the perfume raw material class assignments are stored in an Excel spreadsheet. The data is imported into the JMP™ statistical program. The recursive partitioning method is used to develop classifier model based on the test materials. The model can be is further validated using an external prediction set. The following model is generated by the aforementioned process:
1) Any perfume raw material comprising an ester moiety (PRMCAEM) is assigned to the Stable class if the descriptor S5PC has a value of 1.79 or greater.
2) If the value of S5PC is less than 1.79, and the value of the descriptor FPSA-1 is less than 0.847, the (PRMCAEM) is assigned to the Unstable class.
3) If the value of S5PC is less than 1.79, and the value of the descriptor FPSA-1 is greater than or equal to 0.847, the (PRMCAEM) is assigned to the Uncertain class.
   where: S5PC is the simple fifth-order path-cluster molecular connectivity index computed as described in Kier, L. B.; Hall, L. H.; “Molecular Connectivity in Chemistry and Drug Research;” Academic: New York, 1976. FPSA-1 is the type-1 fractional positive surface area computed as described in “Anal. Chem. 1990, 62,” 2323-2329.

The model is applied and predicts that the following PRMs are useful in laundry formulations in the presence of lipase: bornyl isobutyrate; trans-decahydro-2-naphthyl isobutyrate; 4-allyl-2-methoxyphenyl benzoate; 1-isopropyl-4-methylcyclohex-2-yl acetate; 1-isopropyl-4-methylcyclohex-2-yl proprionate; isopropyl nicotinate; 4-tert-butylcyclohexyl isobutyrate; p-Menth-1-en-8-yl 3-phenylpropenoate; o-tolyl isobutyrate; 3-Butyl-5-methyltetrahydro-2H-pyranyl-4 acetate.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A modeling method for designing health and beauty consumer products, comprising:
a.) correlating a dependent property of an initial consumer product component with an independent variable of said component;
b.) calculating said dependent property for an additional consumer product component by inputting said independent variable of said additional consumer product component into the correlation of Step a.)
c.) optionally, using the output of Step b.) to refine the correlation of Step a.); and
d.) optionally repeating Steps a.) through c.).
